(19) United States
(12) Patent Application Publication
(10) Pub. No.: US 2003/0186997 A1
(43) Pub. Date: Oct. 2, 2003

(54) 2,5-Dihydro-pyrazolo[3,4-d]pyrimidin-4-ones with an anticonsulsive action and methods for producing the same

(76) Inventors: Thomas Arnold, Radebeul (DE); Hans-Joachim Lankau, Weinbohla (DE); Klaus Unverferth, Dresden (DE); Christine Tober, Weinbohla (DE); Chris Rundfeldt, Coswig (DE); Rita Dost, Dresden (JP); Katrin Bernoster, Radebeul (JP); Antje Gasparic, Coswig (JP)

Correspondence Address: FULBRIGHT & JAWORSKI, LLP, 666 FIFTH AVE, NEW YORK, NY, 10103-3198, US

(21) Appl. No.: 10/333,504

(22) PCT Filed: Aug. 24, 2001
(86) PCT No.: PCT/EP01/09811

(30) Foreign Application Priority Data
Aug. 26, 2000 (DE) 100 42 092.3

(51) Int. Cl. A61K 31/519; C07D 487/02

(52) U.S. Cl. 514/262.1; 544/262

(57) ABSTRACT

The invention relates to 2,5-dihydropyrazolo[3,4-d]pyrimidin-4-ones and their tautomers which contain in the 5-position an ar(alkyl) radical and in the 2-position a hydrogen or an ar(alkyl) radical, processes for their preparation and their use as medicaments, in particular for the treatment of epilepsy of various forms.

### TECHNICAL FIELD

The invention relates to 2,5-dihydropyrazolo-[3,4-d]pyrimidin-4-ones and their tautomers which contain an ar(alkyl) radical in the 5-position and a hydrogen or an ar(alkyl) radical in the 2-position, processes for their preparation and their use as medicaments, in particular for the treatment of epilepsy of various forms. On account of the structural similarities to adenine, pyrazolo[3,4-d]pyrimidines are compounds of pharmacological interest.

### PRIOR ART

Hitherto, only 5-benzyl-2,5-dihydropyrazolo[3,4-d]pyrimidin-4-one and 5-phenethyl-2,5-dihydropyrazolo[3,4-d]pyrimidin-4-one have been described [Sochneva, E. O.; Solov'eva, N. P.; Granik, V. G., Khim. Geterotsikl. Soedin. 1978, (12), 1671-6; Granik, V. G.; Sochneva, E. O.; Solov'eva, N. P.; Shvarts, G. Ya.; Syubaev, R. D.; Mashkovskii, M. D., Khim.-Farm. Zh. 1980, 14(6), 36-40]. These compounds were investigated for antiinflammatory action; an anticonvulsant action is not mentioned or suggested.

5-Arylmetyl-2,5-dihydropyrazolo[3,4-d]pyrimidin-4-ones [sic] which have a further substituent in the pyrazole ring are not known.

Known anticonvulsants on the one hand have the disadvantage that undesired side effects, such as neurotoxicity and idiosyncrasies, occur and on the other hand these are not active in certain forms of epilepsy.

The invention is therefore based on the object of making available, compounds having favorable pharmacological properties which have anticonvulsant activity and can be employed as medicaments, in particular for the treatment of epilepsy.

### DESCRIPTION OF THE INVENTION

According to the present invention, these novel compounds are 2,5-dihydropyrazolo[3,4-d]pyrimidin-4-ones of the general formula 1

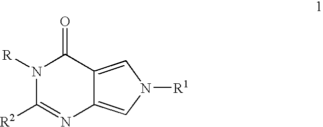

or their tautomers, where

R=CH₂-phenyl, in which phenyl can be mono- or polysubstituted by halogen, C₁-C₃-alkyl, straight-chain or branched, optionally mono- or polysubstituted by halogen C₁-C₃-alkyloxy, straight-chain or branched phenyl NO₂ CN; CH₂-pyridinyl

R¹=H; C₁-C₄-alkyl; phenyl; CH₂-phenyl, in which phenyl can optionally be substituted by halogen; CH₂-pyridinyl; tetrahydrofuranylmethyl

R²=H, methyl excluding the compound in which R is CH₂-phenyl and R₁ is hydrogen.

Examples of compounds of the general formula 1 which may be mentioned are:

5-(2-chlorobenzyl)-2,5-dihydropyrazolo[3,4-d]pyrimidin-4-one

5-(4-chlorobenzyl)-2,5-dihydropyrazolo[3,4-d]pyrimidin-4-one

5-(4-fluorobenzyl)-2,5-dihydropyrazolo[3,4-d]pyrimidin-4-one

5-(2,4-dichlorobenzyl)-2,5-dihydropyrazolo[3,4-d]-pyrimidin-4-one

5-(2,6-difluorobenzyl)-2,5-dihydropyrazolo[3,4-d]-pyrimidin-4-one

5-(2-methylbenzyl)-2,5-dihydropyrazolo[3,4-d]pyrimidin-4-one

5-(2-methoxybenzyl)-2,5-dihydropyrazolo[3,4-d]-pyrimidin-4-one

5-(2-trifluoromethylbenzyl)-2,5-dihydropyrazolo[3,4-d]-pyrimidin-4-one

5-(2,4,6-trimethylbenzyl)-2,5-dihydropyrazolo[3,4-d]-pyrimidin-4-one

5-(2-pyridinylmethyl)-2,5-dihydropyrazolo[3,4-d]-pyrimidin-4-one

5-(3-pyridinylmethyl)-2,5-dihydropyrazolo[3,4-d]-pyrimidin-4-one

5-(4-pyridinylmethyl)-2,5-dihydropyrazolo[3,4-d]-pyrimidin-4-one

5-benzyl-2-methyl-2,5-dihydropyrazolo[3,4-d]pyrimidin-4-one

5-(2-methylbenzyl)-2-methyl-2,5-dihydropyrazolo[3,4-d]-pyrimidin-4-one

5-(3-methylbenzyl)-2-methyl-2,5-dihydropyrazolo[3,4-d]-pyrimidin-4-one

5-(4-methylbenzyl)-2-methyl-2,5-dihydropyrazolo[3,4-d]-pyrimidin-4-one

5-(2-methoxybenzyl)-2-methyl-2,5-dihydropyrazolo[3,4-d]-pyrimidin-4-one

5-(2-chlorobenzyl)-2-methyl-2,5-dihydropyrazolo[3,4-d]-pyrimidin-4-one

5- (3-chlorobenzyl) -2-methyl-2, 5-dihydropyrazolo[3, 4-d]-pyrimidin-4-one

5-(2-trifluoromethylbenzyl)-2-methyl-2,5-dihydropyrazolo[3,4-d]pyrimidin-4-one

5-(2,6-difluorobenzyl)-2-methyl-2,5-dihydropyrazolo[3,4-d]pyrimidin-4-one

5-(4-trifluoromethylbenzyl)-2-methyl-2,5-dihydropyrazolo[3,4-d]pyrimidin-4-one

5-(2-chloro-6-fluorobenzyl)-2-methyl-2,5-dihydropyrazolo[3,4-d]pyrimidin-4-one

5-(2-phenylbenzyl)-2-methyl-2,5-dihydropyrazolo[3,4-d]-pyrimidin-4-one

5-(2,6-dichlorobenzyl)-2-methyl-2,5-dihydro-pyrazolo[3,4-d]pyrimidin-4-one

5-(2-nitrobenzyl)-2-methyl-2,5-dihydropyrazolo[3,4-d]-pyrimidin-4-one

5-(4-chlorobenzyl)-2-methyl-2,5-dihydropyrazolo[3,4-d]-pyrimidin-4-one

5-(2,4-dichlorobenzyl)-2-methyl-2,5-dihydropyrazolo[3,4-d]pyrimidin-4-one

5-(2-iodobenzyl)-2-methyl-2,5-dihydropyrazolo[3,4-d]-pyrimidin-4-one

5-(2-cyanobenzyl)-2-methyl-2,5-dihydropyrazolo[3,4-d]-pyrimidin-4-one

5-(4-fluorobenzyl)-2-methyl-2,5-dihydropyrazolo[3,4-d]-pyrimidin-4-one

5-(2,4,6-trimethylbenzyl)-2-methyl-2,5-dihydropyrazolo[3,4-d]pyrimidin-4-one

5-(2-pyridinylmethyl)-2-methyl-2,5-dihydropyrazolo[3,4-d]-pyrimidin-4-one

5-(4-pyridinylmethyl)-2-methyl-2,5-dihydropyrazolo[3, 4-d]-pyrimidin-4-one

5-(2-chloro-6-fluorobenzyl)-2-ethyl-2,5-dihydropyrazolo[3,4-d]pyrimidin-4-one

5-(4-methylbenzyl)-2-ethyl-2,5-dihydropyrazolo[3,4-d]-pyrimidin-4-one

5-(2,6-difluorobenzyl)-2-ethyl-2,5-dihydropyrazolo[3,4-d]-pyrimidin-4-one

5-(2-pyridinylmethyl)-2-ethyl-2,5-dihydropyrazolo[3,4-d]-pyrimidin-4-one

5-(2-chloro-6-fluorobenzyl)-2-propyl-2,5-dihydropyrazolo[3,4-d]pyrimidin-4-one

5-(4-methylbenzyl)-2-propyl-2,5-dihydropyrazolo[3,4-d]-pyrimidin-4-one

5-(2,6-difluorobenzyl)-2-propyl-2,5-dihydropyrazolo[3,4-d]pyrimidin-4-one

5-(2-pyridinylmethyl)-2-propyl-2,5-dihydropyrazolo[3,4-d]-pyrimidin-4-one

5-(2-chloro-6-fluorobenzyl)-2-isopropyl-2,5-dihydropyrazolo[3,4-d]pyrimidin-4-one

5-(4-methylbenzyl)-2-isopropyl-2,5-dihydropyrazolo[3,4-d]-pyrimidin-4-one

5-(2,6-difluorobenzyl)-2-isopropyl-2,5-dihydropyrazolo[3,4-d]pyrimidin-4-one

5-(2-pyridinylmethyl)-2-isopropyl-2,5-dihydropyrazolo[3,4-d]pyrimidin-4-one

5-(2-chloro-6-fluorobenzyl)-2-butyl-2,5-dihydropyrazolo[3,4-d]pyrimidin-4-one

5-(4-methylbenzyl)-2-butyl-2,5-dihydropyrazolo[3,4-d]-pyrimidin-4-one

5-(2,6-difluorobenzyl)-2-butyl-2,5-dihydropyrazolo[3,4-d]-pyrimidin-4-one

5-(2-chloro-6-fluorobenzyl)-2-phenyl-2,5-dihydropyrazolo[3,4-d]pyrimidin-4-one

5-(4-methylbenzyl)-2-phenyl-2,5-dihydropyrazolo[3,4-d]-pyrimidin-4-one

5-(2,6-difluorobenzyl)-2-phenyl-2,5-dihydropyrazolo[3,4-d]pyrimidin-4-one

5-(2-pyridinylmethyl)-2-phenyl-2,5-dihydropyrazolo[3,4-d]-pyrimidin-4-one

5-(2-chloro-6-fluorobenzyl)-2-benzyl-2,5-dihydropyrazolo[3,4-d]pyrimidin-4-one

5-(4-methylbenzyl)-2-benzyl-2,5-dihydropyrazolo[3,4-d]-pyrimidin-4-one

5-(2,6-difluorobenzyl)-2-benzyl-2,5-dihydropyrazolo[3,4-d]pyrimidin-4-one

5-(2-pyridinylmethyl)-2-benzyl-2,5-dihydropyrazolo[3,4-d]-pyrimidin-4-one

2,5-bis(2-chlorobenzyl)-2,5-dihydropyrazolo[3,4-d]-pyrimidin-4-one

5-(2,6-difluorobenzyl)-2-pyridin-2-ylmethyl-2,5-dihydropyrazolo[3,4-d]pyrimidin-4-one

5-(2-chlorobenzyl)-2-tetrahydrofuran-2-ylmethyl-2,5-dihydropyrazolo[3,4-d]pyrimidin-4-one

5-(2-chlorobenzyl)-6-methyl-2,5-dihydropyrazolo[3,4-d]-pyrimidin-4-one

5-(2,6-difluorobenzyl)-6-methyl-2,5-dihydropyrazolo[3,4-d]hpyrimidin-4-one

5-(2-trifluoromethylbenzyl)-2,6-dimethyl-2,5-dihydropyrazolo[3,4-d]pyrimidin-4-one

5-(2,6-difluorobenzyl)-2,6-dimethyl-2,5-dihydropyrazolo[3,4-d]pyrimidin-4-one

The process for the preparation of compounds of the formula 1 and their tautomers starts from known 3-aminopyrazol-4-carboxylic acid esters (compounds of the general formula 2) or 3-aminopyrazole-4-carboxamides (compounds of the general formula 3) [P. Schmidt, J. Druey, Helv. Chim. Acta. 1956, 39, 986-991; K. Eichenberger, P. Schmidt, M. Wilhelm, J. Druey; Helv. Chim. Acta 1959, 42, 349-359; J. K. Chakrabarti, T. M. Hotten, I. A. Pullar, N. C. Nicholas, J. Med. Chem. 1989, 32(12), 2573-2582],

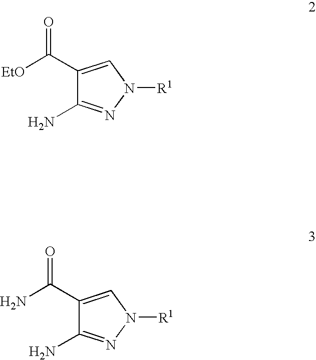

where

R¹=H, C₁-C₄-alkyl; phenyl; CH₂-phenyl, in which phenyl can optionally be substituted by halogen; CH₂-pyridinyl, tetrahydrofuranylmethyl and Et is an alkyl radical.

These compounds of the general formula 2 or general formula 3 are on the one hand cyclized using formamide (R²=H) or acetamide (R²=methyl) at relatively high temperatures, alternatively compounds of the general formula 3 are cyclized using orthoformic acid esters and/or formic acid/acetic anhydride mixtures (R²=H) or using orthoacetic acid ester and/or acetic anhydride (R²=methyl), and then reacted with R-halides, where R has the meaning mentioned, to give compounds of the general formula 1 (Method B).

On the other hand, compounds of the general formula 3 are reacted with dimethylformamide dimethyl acetal (R²=H) or dimethylacetamide dimethyl acetal (R²=methyl) and the products thus obtained are reacted with R-amines, where R has the meaning mentioned, to give compounds of the general formula 1 (Method A).

The compounds according to the invention, just like the already described compound 5-benzyl-2,5-dihydropyrazolo[3,4-d]pyrimidin-4-one, or their pharmaceutically utilizable salts are suitable for the production of pharmaceutical compositions. The pharmaceutical compositions or medicaments can contain one or more of the compounds according to the invention. The customary pharmaceutical vehicles and excipients can be used for the production of the pharmaceutical preparations. The medicaments can be administered, for example, parenterally (e.g. intravenously, intramuscularly, subcutaneously) or orally.

The administration forms can be prepared by the processes which are generally known and customary in pharmaceutical practice.

The compounds according to the invention have strong anticonvulsant actions, just like the already described compound 5-benzyl-2,5-dihydropyrazolo[3,4-d]pyrimidin-4-one.

Anticonvulsant Activity

The compounds according to the invention were tested for their anticonvulsant action in vivo after i.p. administration to mice according to the international customary standard (Pharmac. Weekblad, Sc. Ed. 14, 132 (1992) and Antiepileptic Drugs, Third Ed., Raven Press, New York 1989) (Table 1).

TABLE 1
Anticonvulsant action of selected 2,5-
dihydropyrazolo[3, 4-d]pyrimidin-4-ones

| Compound¹⁾ | log p²⁾ | Test³⁾ | dose⁴⁾ | Action⁵⁾ |
|---|---|---|---|---|
| 1 | 1.14 | MES | 30 | 100 |
| | | PTZ | 100 | 80 |
| 2 | 1.34 | MES | 100 | 33 |
| | | PTZ | 100 | — |
| 3 | 0.62 | MES | 100 | 33 |
| | | PTZ | 300 | 60 |
| 4 | 1.96 | MES | 100 | 100 |
| | | PTZ | 300 | 40 |
| 5 | 0.28 | MES | 30 | 100 |
| | | PTZ | 300 | — |
| 6 | 1.36 | MES | 100 | 67 |
| | | PTZ | 300 | 100 |
| 8 | 0.78 | MES | 100 | 67 |
| | | PTZ | 100 | 20 |
| 13 | 0.72 | MES | 100 | 100 |
| | | PTZ | 300 | — |
| 14 | 1.76 | MES | 30 | 100 |
| | | PTZ | 100 | 80 |
| 17 | 0.92 | MES | 100 | 100 |
| | | PTZ | 300 | 100 |
| 19 | 1.56 | MES | 100 | 100 |
| | | PTZ | 300 | — |
| 21 | 0.67 | MES | 30 | 100 |
| | | PTZ | 100 | 80 |
| 37 | 1.43 | MES | 100 | 80 |
| | | PTZ | 300 | 20 |
| 43 | 1.88 | MES | 300 | 100 |
| | | PTZ | 300 | 33 |
| 58 | 2.59 | MES | 300 | — |
| | | PTZ | 300 | — |
| 60 | 1.67 | MES | 100 | 67 |
| | | PTZ | 300 | — |
| 5-Benzyl-2,5- | 0.47 | MES | 30 | 100 |
| dihydropyrazolo[3,4- | | PTZ | 30 | 100 |
| d]pyrimidin-4-one | | | | |
| Comparison Substances | | | | |

| | | | |
|---|---|---|---|
| Carbamazepine | MES | 100 | 100 |
| | PTZ | 100 | 0 |
| Valproate | MES | 100 | 0 |
| | PTZ | 100 | 30 |

Notes for Table 1:
^{1) Numbering of the compounds according to the examples in Table 2}
^{2) Octanol/water partition coefficient}
^{3) Mouse i.p.: MES = maximal electroshock, PTZ = s.c. pentetrazole}
^{4) in mg/kg}
^{5) in % of the protected animals}

Analogous results were obtained for the oral action.

For example, for compound 1, (5-(2-chlorobenzyl)-2,5-dihydropyrazolo[3,4-d]pyrimidin-4-one), in the rat in maximal electroshock the ED₅₀ (p.o.) was determined to be 18 mg/kg and for the neurotoxicity the NT₅₀ to be >500 mg/kg. This compound is also active in convulsion models using bicuculline and picrotoxin as convulsion-inducing cause. Compound 14 (5-(2-methylbenzyl)-2-methyl-2,5-dihydropyrazolo[3,4-d]pyrimidin-4-one) is likewise strongly anticonvulsant with a large therapeutic breadth (ED₅₀ (rat p.o.) =9 mg/kg, NT₅₀>300 mg/kg). Compound 21 has a similar pharmacological profile (ED₅₀ (rat p.o.) =3 mg/kg, NT₅₀>300 mg/kg).

Working Examples

The following examples serve to illustrate the invention further without restricting it to these.

General Procedure for the Preparation of the Compounds of the Formula 1 and their Tautomers as in Table 2 (Method A)

50 mmol of 3-aminopyrazole-4-carboxamide are reacted with dimethylformamidedimethyl acetal (R²=H) or dimethylacetamide dimethyl acetal (R²=methyl) at relatively high temperature, preferably 90-130° C., in/or without an organic solvent. After 12-40 h, excess solvent and reagent is [sic] completely removed. 50 mmol of an R-amine, in which R has the meaning mentioned, are added to the residue and, if appropriate, an inert organic solvent, preferably xylene, chlorobenzene etc. The reaction mixture is reacted at relatively high temperature, preferably 100-180° C. After 10-35 h, the solvent is removed and the compound of the formula 1 is obtained pure by recrystallization from an organic solvent, preferably DMF, ethanol, methanol or acetone, or alternatively by chromatography.

General Procedure for the Preparation of the Compounds of the Formula 1 and their Tautomers as in Table 2 (Method B)

1^{st} stage

50 mmol of 3-aminopyrazole-4-carboxylic acid ester/amide are reacted at relatively high temperatures (100-200° C.) for 3-15 hours in formamide (R²=H) or acetamide (R²=methyl). After completion of the reaction, the products (pyrazolopyrimidines) are either isolated by filtration, or recovered by chromatography after the removal of the solvent.

Alternatively, 50 mmol of 3-aminopyrazole-4-carboxamide are reacted with orthoformic acid ester and/or with formic acid/acetic anhydride mixture (R²=H) or with orthoacetic acid esters and/or with acetic anhydride (R²=methyl) for 10-50 h at relatively high temperature, preferably 80-120° C. After completion of the reaction, the products (pyrazolopyrimidines) are either isolated by filtration, or recovered by chromatography after the removal of the solvent.

2^{nd} stage

20 mol of pyrazolopyrimidine are dissolved in DMF, treated with an inorganic base, preferably sodium, potassium or calcium carbonate, and sodium/potassium iodide and reacted with 25-40 mmol of an R-halide, where R has the meaning mentioned, at relatively high temperature, preferably 50-140° C. After 5-40 h, the reaction mixture is filtered and the compound of the formula 1 is either isolated by filtration, or recovered by chromatography after the removal of the solvent. The crude products thus obtained are recrystallized from an organic solvent, preferably DMF, ethanol, methanol or acetone. Alternatively, purification can be carried out by chromatography.

TABLE 2
Pyrazolo[3,4-d]pyrimidines

| | | | | Yield | M.p. | |
|---|---|---|---|---|---|---|
| Compound | R ¹⁾ | R^{1 1)} | R^{2 1)} | in (%) | (° C.) | Method |
| 1 | 2-Cl-Bn | H | H | 38 | 186-187 | A |
| 2 | 4-Cl-Bn | H | H | 41 | 246-247 | A |
| 3 | 4-F-Bn | H | H | 84 | 260-261 | A |
| 4 | 2,4-Cl₂-Bn | H | H | 63 | 236-237 | A |
| 5 | 2,6-F₂-Bn | H | H | 15 | 214-216 | A |
| 6 | 2-Me-Bn | H | H | 46 | 221-222 | A |
| 7 | 2-MeO-Bn | H | H | 18 | 188-189 | A |
| 8 | 2-CF₃-Bn | H | H | 29 | 168-171 | A |
| 9 | 2,4,6-Me₃-Bn | H | H | 34 | 199-200 | A |
| 10 | 2-Py-CH₂— | H | H | 45 | 224-225 | A |
| 11 | 3-Py-CH₂— | H | H | 39 | 200-201 | A |
| 12 | 4-Py-CH₂— | H | H | 47 | 220-221 | A |
| 13 | Bn | Me | H | 28 | 202-204 | B |
| 14 | 2-Me-Bn | Me | H | 28 | 185-186 | A |
| 15 | 3-Me-Bn | Me | H | 5 | 114-117 | A |
| 16 | 4-Me-Bn | Me | H | 34 | 224-226 | A |
| 17 | 2-MeO-Bn | Me | H | 31 | 160-162 | B |
| 18 | 2-Cl-Bn | Me | H | 25 | 205-206 | A |
| 19 | 3-Cl-Bn | Me | H | 15 | 169-170 | A |
| 20 | 2-CF₃-Bn | Me | H | 23 | 215-217 | A |
| 21 | 2,6-F₂-Bn | Me | H | 13 | 226-228 | A |
| 22 | 4-CF₃-Bn | Me | H | 56 | 211-212 | B |
| 23 | 2-Cl-6-F-Bn | Me | H | 49 | 216-218 | B |
| 24 | 2-Ph-Bn | Me | H | 59 | 189-191 | B |
| 25 | 2,6-Cl₂-Bn | Me | H | 51 | 200-202 | B |
| 26 | 2-NO₂-Bn | Me | H | 24 | 220-222 | B |
| 27 | 4-Cl-Bn | Me | H | 62 | 209-210 | B |
| 28 | 2,4-Cl₂-Bn | Me | H | 60 | 218-219 | B |
| 29 | 2-1-Bn | Me | H | 16 | 187-190 | B |
| 30 | 2-CN-Bn | Me | H | 21 | 198-199 | B |
| 31 | 4-F-Bn | Me | H | 62 | 216-217 | B |
| 32 | 2,4,6-Me₃-Bn | Me | H | 45 | 182-184 | B |
| 33 | 2-Py-CH₂— | Me | H | 53 | 203-205 | B |
| 34 | 4-Py-CH₂— | Me | H | 48 | 192-195 | B |
| 35 | 2-Cl-6-F-Bn | Et | H | 61 | 207-209 | B |
| 36 | 4-Me-Bn | Et | H | 57 | 178-179 | B |
| 37 | 2,6-F₂-Bn | Et | H | 63 | 185-187 | B |
| 38 | 2-Py-CH₂— | Et | H | 71 | 190-191 | B |
| 39 | 2-Cl-6-F-Bn | Pro | H | 73 | 163-165 | B |
| 40 | 4-Me-Bn | Pro | H | 68 | 157-158 | B |
| 41 | 2,6-F₂-Bn | Pro | H | 65 | 167-168 | B |
| 42 | 2-Py-CH₂— | Pro | H | 72 | 179-181 | B |
| 43 | 2-Cl-6-F-Bn | i-Pro | H | 66 | 159-161 | B |
| 44 | 4-Me-Bn | i-Pro | H | 78 | 145-147 | B |
| 45 | 2,6-F₂-Bn | i-Pro | H | 74 | 156-157 | B |
| 46 | 2-Py-CH₂— | i-Pro | H | 63 | 163-164 | B |
| 47 | 2-Cl-6-F-Bn | Bu | H | 45 | 156-157 | B |
| 48 | 4-Me-Bn | Bu | H | 51 | 146-147 | B |
| 49 | 2,6-F₂-Bn | Bu | H | 62 | 151-152 | B |
| 50 | 2-Cl-6-F-Bn | Ph | H | 67 | 195-197 | B |
| 51 | 4-Me-Bn | Ph | H | 71 | 182-183 | B |
| 52 | 2,6-F₂-Bn | Ph | H | 65 | 189-191 | B |
| 53 | 2-Py-CH₂— | Ph | H | 75 | 199-201 | B |
| 54 | 2-Cl-6-F-Bn | Bn | H | 48 | 176-177 | B |
| 55 | 4-Me-Bn | Bn | H | 54 | 167-169 | B |
| 56 | 2,6-F₂-Bn | Bn | H | 58 | 173-174 | B |
| 57 | 2-Py-CH₂— | Bn | H | 44 | 182-184 | B |
| 58 | 2-Cl-Bn | 2-Cl-Bn | H | 42 | 167-168 | B |
| 59 | 2,6-F₂-Bn | 2-Py-CH₂— | H | 49 | 174-175 | B |
| 60 | 2-Cl-Bn | 2-THF—CH₂— | H | 39 | 128-130 | A |
| 61 | 2-Cl-Bn | H | Me | 24 | 222-224 | A |
| 62 | 2,6-F₂-Bn | H | Me | 18 | 235-236 | A |
| 63 | 2-CF₃-Bn | Me | Me | 47 | 192-193 | B |
| 64 | 2,6-F₂-Bn | Me | Me | 54 | 201-202 | B |

¹⁾ Abbreviations used: Me = CH₃, Et = C₂H₅, Pro = C₃H₇, i-Pro = i-C₃H₇, Bu = C₄H₉, Ph = C₆H₅, Bn = —CH₂—C₆H₅, Py = C₈H₄N, THF = C₄H₈O.

Patent claims:

1. A novel compound of the general formula 1 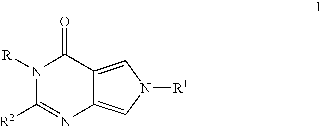
or its tautomers, where
R=CH₂-phenyl, in which phenyl can be mono- or polysubstituted by halogen,
C₁-C₃-alkyl, straight-chain or branched, optionally mono- or polysubstituted by halogen
C₁-C₃-alkyloxy, straight-chain or branched phenyl NO₂ CN CH₂-pyridinyl;
R=H; C₁-C₄-alkyl; phenyl; CH₂-phenyl, in which phenyl can optionally be substituted by halogen; CH₂-pyridinyl; tetrahydrofuranyl-methyl
R²=H, methyl excluding the compound in which R is CH₂-phenyl and R₁ is hydrogen.

2. A compound of the general formula 1
5-(2-chlorobenzyl)-2,5-dihydropyrazolo[3,4-d]pyrimidin-4-one
5-(4-chlorobenzyl)-2,5-dihydropyrazolo[3,4-d]pyrimidin-4-one
5-(4-fluorobenzyl)-2,5-dihydropyrazolo[3,4-d]pyrimidin-4-one
5-(2,4-dichlorobenzyl)-2,5-dihydropyrazolo[3,4-d]pyrimidin-4-one
5-(2,6-difluorobenzyl)-2,5-dihydropyrazolo[3,4-d]pyrimidin-4-one
5-(2-methylbenzyl)-2,5-dihydropyrazolo[3,4-d]pyrimidin-4-one
5-(2-methoxybenzyl)-2,5-dihydropyrazolo[3,4-d]pyrimidin-4-one
5-(2-trifluoromethylbenzyl)-2,5-dihydropyrazolo[3,4-d]pyrimidin-4-one
5-(2,4,6-trimethylbenzyl)-2,5-dihydropyrazolo[3,4-d]pyrimidin-4-one
5-(2-pyridinylmethyl)-2,5-dihydropyrazolo[3,4-d]pyrimidin-4-one
5-(3-pyridinylmethyl)-2,5-dihydropyrazolo[3,4-d]pyrimidin-4-one
5-(4-pyridinylmethyl)-2,5-dihydropyrazolo[3,4-d]pyrimidin-4-one
5-benzyl-2-methyl-2,5-dihydropyrazolo[3,4-d]pyrimidin-4-one
5-(2-methylbenzyl)-2-methyl-2,5-dihydropyrazolo[3,4-d]pyrimidin-4-one
5-(3-methylbenzyl)-2-methyl-2,5-dihydropyrazolo[3,4-d]pyrimidin-4-one
5-(4-methylbenzyl)-2-methyl-2,5-dihydropyrazolo[3,4-d]pyrimidin-4-one
5-(2-methoxybenzyl)-2-methyl-2,5-dihydropyrazolo[3,4-d]pyrimidin-4-one
5-(2-chlorobenzyl)-2-methyl-2,5-dihydropyrazolo[3,4-d]pyrimidin-4-one
5-(3-chlorobenzyl)-2-methyl-2,5-dihydropyrazolo[3,4-d]pyrimidin-4-one
5-(2-trifluoromethylbenzyl)-2-methyl-2,5-dihydropyrazolo[3,4-d]pyrimidin-4-one
5-(2,6-difluorobenzyl)-2-methyl-2,5-dihydropyrazolo[3,4-d]pyrimidin-4-one
5-(4-trifluoromethylbenzyl)-2-methyl-2,5-dihydropyrazolo[3,4-d]pyrimidin-4-one
5-(2-chloro-6-fluorobenzyl)-2-methyl-2,5-dihydropyrazolo[3,4-d]pyrimidin-4-one
5-(2-phenylbenzyl)-2-methyl-2,5-dihydropyrazolo[3,4-d]pyrimidin-4-one
5-(2,6-dichlorobenzyl)-2-methyl-2,5-dihydropyrazolo[3,4-d]pyrimidin-4-one
5-(2-nitrobenzyl)-2-methyl-2,5-dihydropyrazolo[3,4-d]pyrimidin-4-one
5-(4-chlorobenzyl)-2-methyl-2,5-dihydropyrazolo[3,4-d]pyrimidin-4-one
5-(2,4-dichlorobenzyl)-2-methyl-2,5-dihydropyrazolo[3,4-d]pyrimidin-4-one
5-(2-iodobenzyl)-2-methyl-2,5-dihydropyrazolo[3,4-d]pyrimidin-4-one
5-(2-cyanobenzyl)-2-methyl-2,5-dihydropyrazolo[3,4-d]pyrimidin-4-one
5-(4-fluorobenzyl)-2-methyl-2,5-dihydropyrazolo[3,4-d]pyrimidin-4-one
5-(2,4,6-trimethylbenzyl)-2-methyl-2,5-dihydropyrazolo[3,4-d]pyrimidin-4-one
5-(2-pyridinylmethyl)-2-methyl-2,5-dihydropyrazolo[3,4-d]pyrimidin-4-one
5-(4-pyridinylmethyl)-2-methyl-2,5-dihydropyrazolo[3,4-d]pyrimidin-4-one
5-(2-chloro-6-fluorobenzyl)-2-ethyl-2,5-dihydropyrazolo[3,4-d]pyrimidin-4-one
5-(4-methylbenzyl)-2-ethyl-2,5-dihydropyrazolo[3,4-d]pyrimidin-4-one
5-(2,6-difluorobenzyl)-2-ethyl-2,5-dihydropyrazolo[3,4-d]pyrimidin-4-one
5-(2-pyridinylmethyl)-2-ethyl-2,5-dihydropyrazolo[3,4-d]pyrimidin-4-one
5-(2-chloro-6-fluorobenzyl)-2-propyl-2,5-dihydropyrazolo[3,4-d]pyrimidin-4-one
5-(4-methylbenzyl)-2-propyl-2,5-dihydropyrazolo[3,4-d]pyrimidin-4-one
5-(2,6-difluorobenzyl)-2-propyl-2,5-dihydropyrazolo[3,4-d]pyrimidin-4-one
5-(2-pyridinylmethyl)-2-propyl-2,5-dihydropyrazolo[3,4-d]pyrimidin-4-one
5-(2-chloro-6-fluorobenzyl)-2-isopropyl-2,5-dihydropyrazolo[3,4-d]pyrimidin-4-one
5-(4-methylbenzyl)-2-isopropyl-2,5-dihydropyrazolo[3,4-d]pyrimidin-4-one
5-(2,6-difluorobenzyl)-2-isopropyl-2,5-dihydropyrazolo[3,4-d]pyrimidin-4-one
5-(2-pyridinylmethyl)-2-isopropyl-2,5-dihydropyrazolo[3,4-d]pyrimidin-4-one
5-(2-chloro-6-fluorobenzyl)-2-butyl-2,5-dihydropyrazolo[3,4-d]pyrimidin-4-one
5-(4-methylbenzyl)-2-butyl-2,5-dihydropyrazolo[3,4-d]pyrimidin-4-one
5-(2,6-difluorobenzyl)-2-butyl-2,5-dihydropyrazolo[3,4-d]pyrimidin-4-one
5-(2-chloro-6-fluorobenzyl)-2-phenyl-2,5-dihydropyrazolo[3,4-d]pyrimidin-4-one
5-(4-methylbenzyl)-2-phenyl-2,5-dihydropyrazolo[3,4-d]pyrimidin-4-one
5-(2,6-difluorobenzyl)-2-phenyl-2,5-dihydropyrazolo[3,4-d]pyrimidin-4-one
5-(2-pyridinylmethyl)-2-phenyl-2,5-dihydropyrazolo[3,4-d]pyrimidin-4-one
5-(2-chloro-6-fluorobenzyl)-2-benzyl-2,5-dihydropyrazolo[3,4-d]pyrimidin-4-one
5-(4-methylbenzyl)-2-benzyl-2,5-dihydropyrazolo[3,4-d]pyrimidin-4-one
5-(2,6-difluorobenzyl)-2-benzyl-2,5-dihydropyrazolo[3,4-d]pyrimidin-4-one
5-(2-pyridinylmethyl)-2-benzyl-2,5-dihydropyrazolo[3,4-d]pyrimidin-4-one
2,5-bis(2-chlorobenzyl)-2,5-dihydropyrazolo[3,4-d]pyrimidin-4-one
5-(2,6-difluorobenzyl)-2-pyridin-2-ylmethyl-2,5-dihydropyrazolo[3,4-d]pyrimidin-4-one
5-(2-chlorobenzyl)-2-tetrahydrofuran-2-ylmethyl-2,5-dihydropyrazolo[3,4-d]pyrimidin-4-one
5-(2-chlorobenzyl)-6-methyl-2,5-dihydropyrazolo[3,4-d]pyrimidin-4-one
5-(2,6-difluorobenzyl)-6-methyl-2,5-dihydropyrazolo[3,4-d]pyrimidin-4-one
5-(2-trifluoromethylbenzyl)-2,6-dimethyl-2,5-dihydropyrazolo[3,4-d]pyrimidin-4-one
5-(2,6-difluorobenzyl)-2,6-dimethyl-2,5-dihydropyrazolo[3,4-d]pyrimidin-4-one

3. A process for the preparation of 2,5-dihydropyrazolo[3,4-d]pyrimidin-4-ones of the general formula 1, which comprises reacting compounds of the general formula 3, with dimethylformamide dimethyl acetal or with dimethylacetamide dimethyl acetal and then cyclizing with R-amines, where R has the meaning mentioned (Method A).

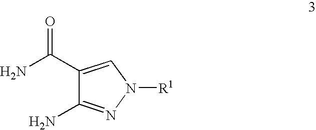

4. A process for the preparation of 2,5-dihydropyrazolo[3,4-d]pyrimidin-4-ones of the general formula 1, which comprises cyclizing compounds of the general formula 2, where Et is an alkyl radical, or of the general formula 3 with formamide or acetamide and then reacting with R-halides, where R has the meaning mentioned (Method B).

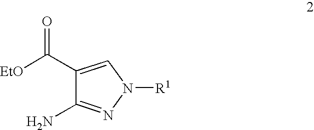

5. The process as claimed in claim 4, wherein, for the cyclization of compounds of the general formula 3, orthoformic acid esters and/or formic acid/acetic anhydride mixtures or orthoacetic acid esters and/or acetic anhydride are used.

6. A pharmaceutical composition which contains one or more 2,5-dihydropyrazolo[3,4-d]pyrimidin-4-ones of the formula 1 as claimed in claim 1 or their pharmaceutically utilizable salts as active compounds in addition to one or more physiologically tolerable excipients and/or vehicles and, if appropriate, a diluent.

7. A pharmaceutical composition which comprises one or more 2,5-dihydropyrazolo[3,4-d]pyrimidin-4-ones as claimed in claim 2 or their pharmaceutically utilizable salts as active compounds in addition to one or more physiologically tolerable excipients and/or vehicles and, if appropriate, a diluent.

8. A pharmaceutical composition which comprises 5-benzyl-2,5-dihydropyrazolo[3,4-d]pyrimidin-4-one or its pharmaceutically utilizable salts as active compounds in addition to one or more physiologically tolerable excipients and/or vehicles and, if appropriate, a diluent.

9. A pharmaceutical composition which comprises 5-benzyl-2,5-dihydropyrazolo[3,4-d]pyrimidin-4-one and one or more 2,5-dihydropyrazolo[3,4-d]pyrimidin-4-ones as claimed in claim 1 or claim 2 or their pharmaceutically utilizable salts as active compounds in addition to one or more physiologically tolerable excipients and/or vehicles and, if appropriate, a diluent.

10. The use of 2,5-dihydropyrazolo[3,4-d]pyrimidin-4-ones as in formula 1 for the production of medicaments for the treatment of diseases [lacuna] of epilepsy of various forms.

11. The use of 5-benzyl-2,5-dihydropyrazolo[3,4-d]pyrimidin-4-one for the production of medicaments for the treatment of diseases [lacuna] of epilepsy of various forms.
